(19) United States
(12) Patent Application Publication
(10) Pub. No.: US 2025/0303199 A1
(43) Pub. Date: Oct. 2, 2025

(54) ULTRASOUND GENERATING DEVICE, METHOD, AND PROGRAM FOR IRRADIATING PARASYMPATHETIC NERVE WITH ULTRASOUND WAVE

(71) Applicant: SKINGRAB CO., LTD., Seoul (KR)

(72) Inventor: Sugun LEE, Paju-si (KR)

(73) Assignee: SKINGRAB CO., LTD., Seoul (KR)

(21) Appl. No.: 19/236,274

(22) Filed: Jun. 12, 2025

Related U.S. Application Data
(63) Continuation of application No. PCT/KR2023/020561, filed on Dec. 13, 2023.

(30) Foreign Application Priority Data
Dec. 13, 2022 (KR) 10-2022-0173442

(51) Int. Cl. A61N 7/00

(52) U.S. Cl. CPC A61N 7/00; A61N 2007/0026

(57) ABSTRACT

The present disclosure includes: an ultrasound generating unit for irradiating the parasympathetic nerve with ultrasound waves; and a control unit for controlling the operation of the ultrasound generating unit, wherein the control unit controls the ultrasound generating unit so that the ultrasound generating unit applies ultrasound energy to a body part corresponding to the parasympathetic nerve.

### CROSS-REFERENCE TO RELATED APPLICATIONS

The present application is a continuation of International Patent Application No. PCT/KR2023/020561, filed on Dec. 13, 2023, which is based upon and claims the benefit of priority to Korean Patent Application No. 10-2022-0173442 filed on Dec. 13, 2022. The disclosures of the above-listed applications are hereby incorporated by reference herein in their entirety.

### BACKGROUND

Embodiments of the present disclosure described herein relate to an ultrasound generating device, and more particularly, relate to a device, a method, and a program for generating ultrasound that irradiate ultrasound to a parasympathetic nerve.

Ultrasound refers to waves with a frequency of 20 KHz or higher, and has the property of penetrating water, and thus it is widely used in the medical field, such as in ultrasonic diagnostic and treatment devices.

The most common application of ultrasound in the medical field is an ultrasound imaging device that utilizes the transmission and reflection properties of ultrasound. For example, there is a device that obtains cross-sectional images of a body by visualizing the time and intensity of reflection of ultrasound as it passes through the body.

However, a conventional ultrasound generating device has limitations in accurately irradiating ultrasound onto each body part. Accordingly, it has limitations in improving surgical accuracy.

Moreover, the conventional ultrasound generating device has limitations in adjusting the ultrasound for each body part under optimal conditions. Accordingly, it has limitations in maximizing surgical outcomes while reducing surgical time.

Therefore, nowadays, various methods have been studied to maximize the surgical outcomes while reducing the surgical time and improving accuracy, by accurately irradiating ultrasound onto each body part and adjusting the irradiation to each body part under the optimal conditions.

### SUMMARY

Embodiments of the present disclosure provide a device that may accurately irradiate ultrasound onto each body part to improve the accuracy of surgery, thereby suppressing at least one of diabetic neuropathy and diabetic peripheral neuropathy.

Embodiments of the present disclosure provide a device that may control ultrasound for each body part under optimal conditions, thereby maximizing the surgical effect while reducing the surgical time.

Problems to be solved by the present disclosure are not limited to the problems mentioned above, and other problems not mentioned will be clearly understood by those skilled in the art from the following description.

According to an embodiment, an ultrasound generating device includes an ultrasound generating unit that irradiates ultrasound onto a parasympathetic nerve, and a control unit that controls an operation of the ultrasound generating unit. The control unit controls the ultrasound generating unit such that the ultrasound generating unit applies ultrasound energy to a body part corresponding to the parasympathetic nerve.

Moreover, the control unit controls a transducer of the ultrasound generating unit such that the ultrasound generating unit adjusts a microvascular blood flow by noninvasively applying ultrasound energy to a body part corresponding to a peripheral nerve.

Furthermore, the control unit controls the transducer of the ultrasound generating unit such that the ultrasound generating unit noninvasively applies ultrasound energy to a certain area in a depth direction of the body part corresponding to the parasympathetic nerve or the peripheral nerve.

Also, the transducer is in contact with a body part of a user having at least one of a flat surface and a curved surface.

In addition, the body part includes at least one of an ear, a neck, an arm, a wrist, a forearm, a hand, a foot, and a leg.

According to an embodiment, a method performed by an ultrasound generating device includes moving an ultrasound generating unit to irradiate ultrasound onto a parasympathetic nerve, and controlling the ultrasound generating unit such that the ultrasound generating unit applies ultrasound energy to a body part corresponding to the parasympathetic nerve when movement of the ultrasound generating unit is completed.

Moreover, the controlling includes controlling a transducer of the ultrasound generating unit such that the ultrasound generating unit adjusts a microvascular blood flow by noninvasively applying ultrasound energy to a body part corresponding to a peripheral nerve.

Furthermore, the controlling includes controlling the transducer of the ultrasound generating unit such that the ultrasound generating unit noninvasively applies ultrasound energy to a certain area in a depth direction of the body part corresponding to the parasympathetic nerve or the peripheral nerve.

Also, the transducer is in contact with a body part of a user having at least one of a flat surface and a curved surface.

Besides, a computer program stored in a computer-readable recording medium for executing a method to implement the present disclosure may be further provided.

In addition, a computer-readable recording medium for recording a computer program for performing the method for implementing the present disclosure may be further provided.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a drawing showing an example of a configuration of an ultrasound generating device, according to an embodiment of the present disclosure;

FIG. 2 is a drawing showing an example of a process in which an ultrasound generating unit irradiates ultrasound;

FIGS. 3 to 6 are drawings showing examples of the shape of a transducer of FIG. 2;

FIGS. 7 to 10 are drawings showing other examples of the shape of the transducer of FIG. 2;

FIGS. 11 to 13 are drawings showing examples of a process in which the ultrasound generating unit of FIG. 1 applies ultrasound energy to a certain area in a depth direction of a body part;

FIG. 14 is a flowchart showing an example of an ultrasound generating method of an ultrasound generating device, according to an embodiment of the present disclosure;

FIG. 15 is a drawing showing another example of a configuration of an ultrasound generating device, according to an embodiment of the present disclosure;

FIG. 16 is a diagram illustrating an example of a process for outputting recommended ultrasound energy intensity and a recommended ultrasound irradiation area, which correspond to at least one of a diabetic neuropathy patient and a diabetic peripheral neuropathy patient, through the processor of FIG. 15;

FIGS. 17 and 18 are flowcharts showing another example of an ultrasound generating method of an ultrasound generating device, according to an embodiment of the present disclosure; and

FIG. 19 is a drawing showing an example of a process of irradiating ultrasound onto a diabetic neuropathy patient or a diabetic peripheral neuropathy patient while an ultrasound generating unit moves by the transfer unit of FIG. 15.

### DETAILED DESCRIPTION

The same reference numerals denote the same elements throughout the present disclosure. The present disclosure does not describe all elements of embodiments. Well-known content in a technical field, to which the present disclosure belongs, or redundant content in which embodiments are the same as one another will be omitted. A term such as ‘unit, module, member, or block’ used in the specification may be implemented with software or hardware. According to embodiments, a plurality of ‘units, modules, members, or blocks’ may be implemented with one component, or a single ‘unit, module, member, or block’ may include a plurality of components.

Throughout this specification, when it is supposed that a portion is “connected” to another portion, this includes not only a direct connection, but also an indirect connection. The indirect connection includes being connected through a wireless communication network.

Furthermore, when a portion “comprises” a component, it will be understood that it may further include another component, without excluding other components unless specifically stated otherwise.

Throughout this specification, when it is supposed that a member is located on another member “on”, this includes not only the case where one member is in contact with another member but also the case where another member is present between two other members.

Terms such as ‘first’, ‘second’, and the like are used to distinguish one component from another component, and thus the component is not limited by the terms described above.

Unless there are obvious exceptions in the context, a singular form includes a plural form.

In each step, an identification code is used for convenience of description. The identification code does not describe the order of each step. Unless the context clearly states a specific order, each step may be performed differently from the specified order.

Hereinafter, operating principles and embodiments of the present disclosure will be described with reference to the accompanying drawings.

First, ultrasound technology is being used not only for diagnosis, treatment, but also for surgery.

This type of ultrasound technology used in surgery may be implemented through an ultrasound generating device. The ultrasound generating device may irradiate ultrasound onto parts of a patient's body.

In this specification, a control unit of an ultrasound generating device according to an embodiment of the present disclosure includes all various devices capable of providing results to a user by performing arithmetic processing. For example, the control unit of the ultrasound generating device according to an embodiment of the present disclosure may include all of a computer, a server device, and a portable terminal, or may be in any one form.

Here, for example, the computer may include a notebook computer, a desktop computer, a laptop computer, a tablet PC, a slate PC, and the like, which are equipped with a web browser.

A server device may be a server that processes information by communicating with an external device and may include an application server, a computing server, a database server, a file server, a mail server, a proxy server, and a web server.

For example, the portable terminal may be a wireless communication device that guarantees portability and mobility, and may include all kinds of handheld-based wireless communication devices such as a smartphone, a personal communication system (PCS), a global system for mobile communication (GSM), a personal digital cellular (PDC), a personal handyphone system (PHS), a personal digital assistant (PDA), International Mobile Telecommunication (IMT)-2000, a code division multiple access (CDMA)-2000, W-Code Division Multiple Access (W-CDMA), and Wireless Broadband Internet terminal (Wibro) terminal, and a wearable device such as a timepiece, a ring, a bracelet, an anklet, a necklace, glasses, a contact lens, or a head-mounted device (HMD).

According to an embodiment of the present disclosure, an ultrasound generating device may accurately irradiate ultrasound onto each body part to improve the accuracy of surgery, thereby suppressing at least one of diabetic neuropathy and diabetic peripheral neuropathy.

Moreover, according to an embodiment of the present disclosure, the ultrasound generating device may adjust and irradiate ultrasound for each body part under optimal conditions, thereby maximizing the surgical effect while reducing the surgical time.

Hereinafter, the ultrasound generating device will be described in detail.

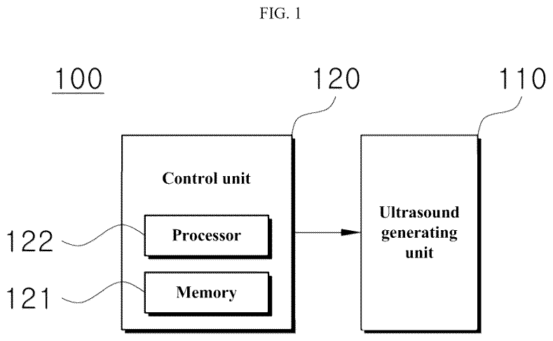

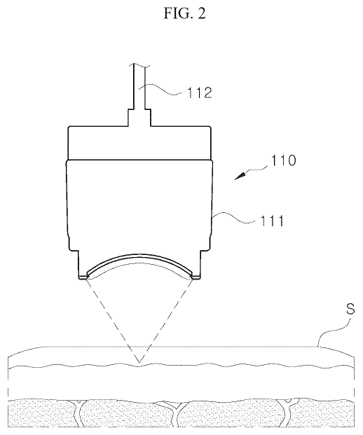

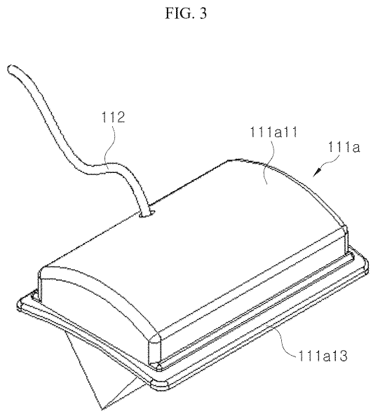

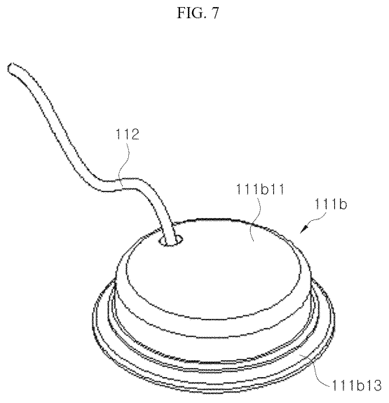

FIG. 1 is a drawing showing an example of a configuration of an ultrasound generating device, according to an embodiment of the present disclosure. FIG. 2 is a drawing showing an example of a process in which an ultrasound generating unit irradiates ultrasound. FIGS. 3 to 6 are drawings showing examples of the shape of a transducer of FIG. 2. FIGS. 7 to 10 are drawings showing other examples of the shape of the transducer of FIG. 2.

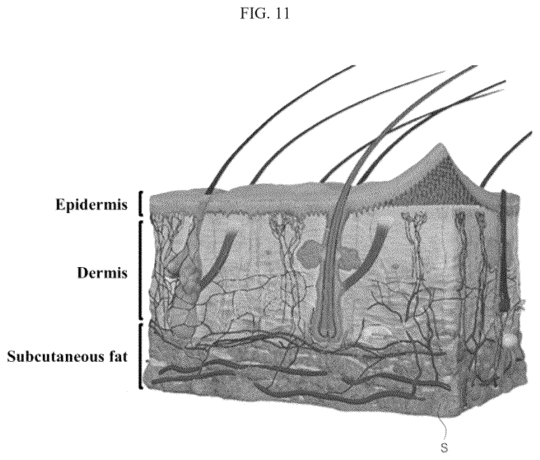

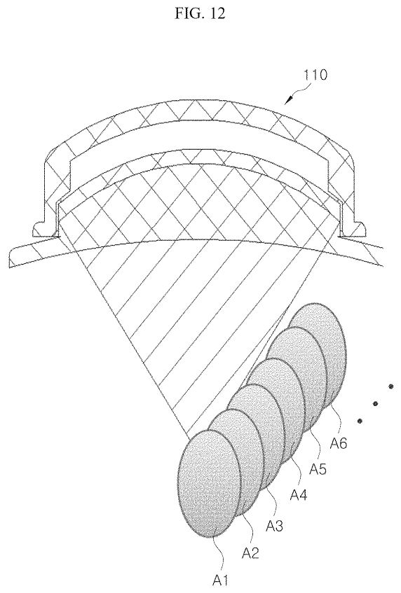

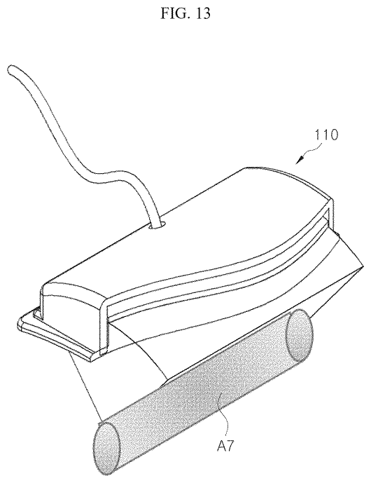

FIGS. 11 to 13 are drawings showing examples of a process in which the ultrasound generating unit of FIG. 1 applies ultrasound energy to a certain area in a depth direction of a body part.

Referring to FIGS. 1 and 2, an ultrasound generating device 100 may include an ultrasound generating unit 110 and a control unit 120.

The ultrasound generating unit 110 having a transducer 111 may irradiate ultrasound onto a body part ‘S’ corresponding to at least one of a parasympathetic nerve or a peripheral nerve. In this case, the peripheral nerve may include at least one of a vagus nerve, an intercostal nerve, a subcostal nerve, an iliohypogastric nerve, an ilioinguinal nerve, a lateral cutaneous femoral nerve, a genitofemoral nerve, a musculocutaneous nerve, a radial nerve, a median nerve, a ulnar nerve, an obturator nerve, a femoral nerve, a muscular branch of a femoral nerve, a saphenous nerve, a sciatic nerve, a tibial nerve, a posterior tibial nerve, a sacral nerve, a common peroneal nerve, a deep peroneal nerve, a superficial peroneal nerve, a sural nerve, a cranial nerve, a spinal cord, a spinal element, a spinal root, a dorsal root ganglion, a sympathetic ganglion, a brachial nerve, or a hair follicle. Here, a power supply unit 112 may include a power cable and may be electrically connected to the transducer 111.

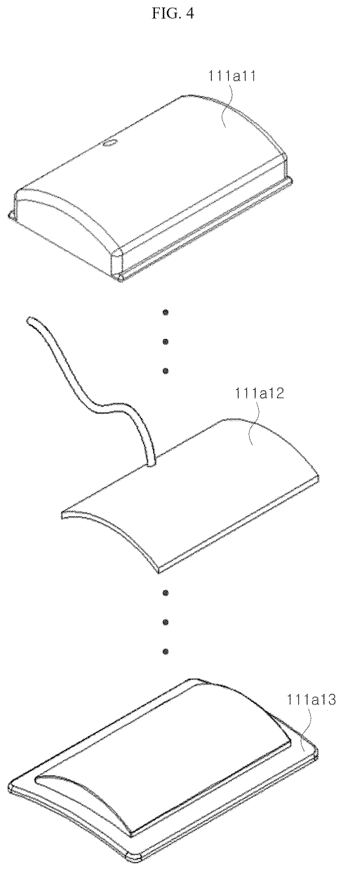

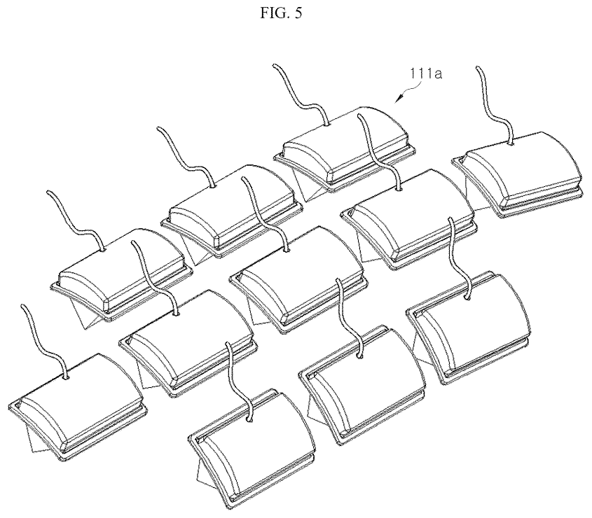

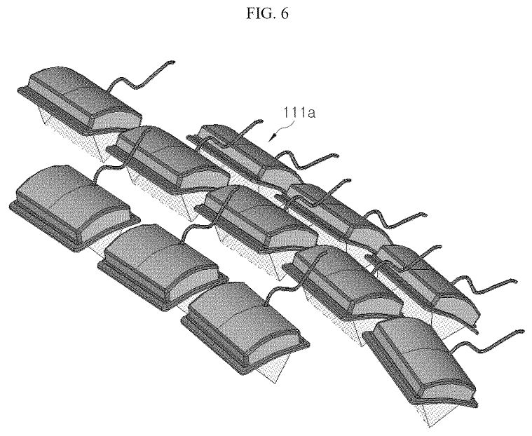

As shown in FIGS. 3 and 4, a transducer 111a may include a cover 111a11, a vibrator 111a12, a gel pad 111a13, and the like. Here, the cover 111a11 may have a linear shape and may be provided to protect the vibrator 111a12; the vibrator 111a12 may have a linear shape and be electrically connected to the power supply unit 112 to generate ultrasound; and the gel pad 111a13 may have a linear shape and may be in contact with the body part ‘S’ of a user having at least one of a flat surface and a curved surface to deliver ultrasound. In this case, as shown in FIGS. 5 and 6, the plurality of transducers 111a may be provided, and each of the plurality of transducers 111a may be in contact with the body part ‘S’ of the user corresponding to at least one of the parasympathetic nerve and the peripheral nerve to irradiate ultrasound. Here, the body part ‘S’ may include at least one of an ear, a neck, an arm, a wrist, a forearm, a hand, a foot, and a leg.

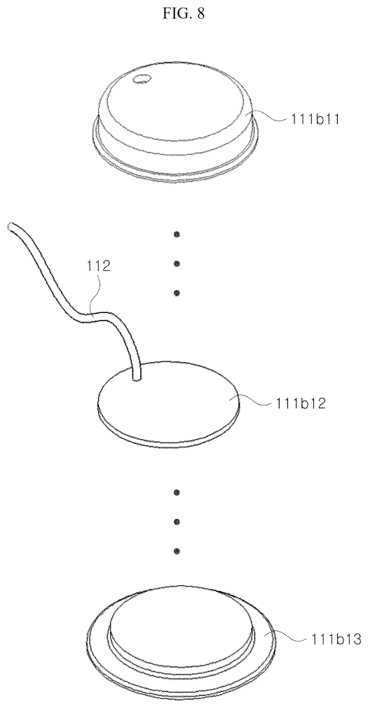

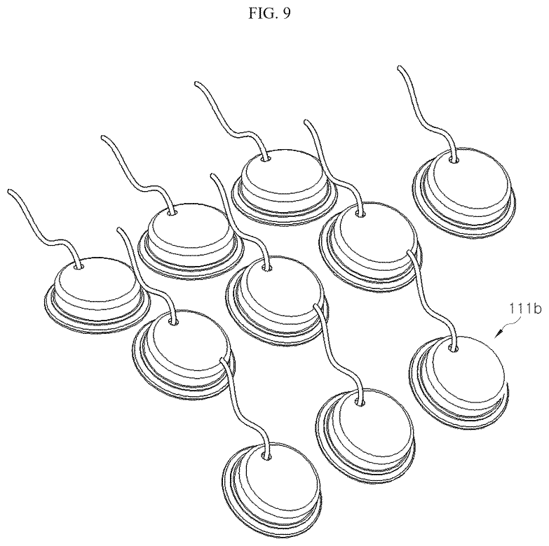

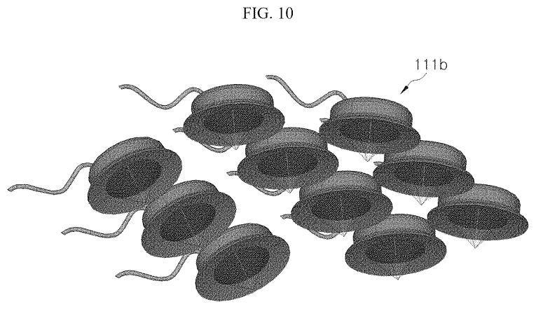

As shown in FIGS. 7 and 8, a transducer 111b may include a cover 111b11, a vibrator 111b12, a gel pad 111b13, and the like. Here, the cover 111b11 may have a cylindrical shape and may be provided to protect the vibrator 111b12; the vibrator 111b12 may have a cylindrical shape and be electrically connected to the power supply unit 112 to generate ultrasound; and the gel pad 111b13 may have a cylindrical shape and may be in contact with the body part ‘S’ of the user having at least one of a flat surface and a curved surface to deliver ultrasound. In this case, as shown in FIGS. 9 and 10, the plurality of transducers 111b may be provided, and each of the plurality of transducers 111b may be in contact with the body part ‘S’ of the user corresponding to at least one of the parasympathetic nerve and the peripheral nerve to irradiate ultrasound. Here, the body part ‘S’ may include at least one of an ear, a neck, an arm, a wrist, a forearm, a hand, a foot, and a leg.

As illustrated in FIGS. 11 to 13, the ultrasound generating unit 110 may adjust a microvascular blood flow by noninvasively applying ultrasound energy to the body part ‘S’ of a user corresponding to at least one of a parasympathetic nerve and a peripheral nerve. In this case, the ultrasound generating unit 110 may adjust the microvascular blood flow by noninvasively applying ultrasound energy to a certain area A1 to A6 . . . , A7 in a depth direction of a user's body part ‘S’ corresponding to at least one of the parasympathetic nerve and the peripheral nerve. Here, the body part ‘S’ may include at least one of an ear, a neck, an arm, a wrist, a forearm, a hand, a foot, and a leg, and the skin layer of the body part ‘S’ may be epidermis, dermis, subcutaneous fat, and the like.

The control unit 120 may be implemented with a memory 121 that stores data regarding an algorithm for controlling operations of components within the present device, or a program for realizing the algorithm, and at least one processor 122 that performs the above-described operation by using the data stored in the memory 121. Here, each of the memory 121 and the processor 122 may be implemented as separate chips. Moreover, the memory 121 and the processor 122 may be implemented as a single chip.

The memory 121 may store data that supports various functions of the present apparatus, and a program for operations of the control unit, may store input/output data, and may store a plurality of application programs (or applications) running on the present apparatus, pieces of data for operations of the present apparatus, and instructions. At least part of the application programs may be downloaded from an external server through wireless communication.

The memory 121 may include the type of a storage medium of at least one of a flash memory type, hard disk type, a solid state disk (SSD) type, a silicon disk drive (SDD) type, a multimedia card micro type, a memory of a card type (e.g., SD memory, XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disc. Furthermore, the memory 121 may be separate from the present apparatus, but may be a database connected by wire or wirelessly.

The processor 122 may control the ultrasound generating unit 110 such that the ultrasound generating unit 110 applies ultrasound energy to the body part ‘S’ corresponding to at least one of a parasympathetic nerve and a peripheral nerve. Here, the processor 122 may allow the transducer 111 of the ultrasound generating unit 110 to adjust a microvascular blood flow by noninvasively applying ultrasound energy to the body part ‘S’ corresponding to the peripheral nerve. At this time, the processor 122 may allow the transducer 111 of the ultrasound generating unit 110 to adjust a microvascular blood flow by non-invasively applying ultrasound energy to a certain area A1 to A6 . . . , A7 in the depth direction of the body part ‘S’ corresponding to at least one of the parasympathetic nerve and the peripheral nerve. Here, the body part ‘S’ may include at least one of an ear, a neck, an arm, a wrist, a forearm, a hand, a foot, and a leg, and the skin layer of the body part ‘S’ may be epidermis, dermis, subcutaneous fat, and the like.

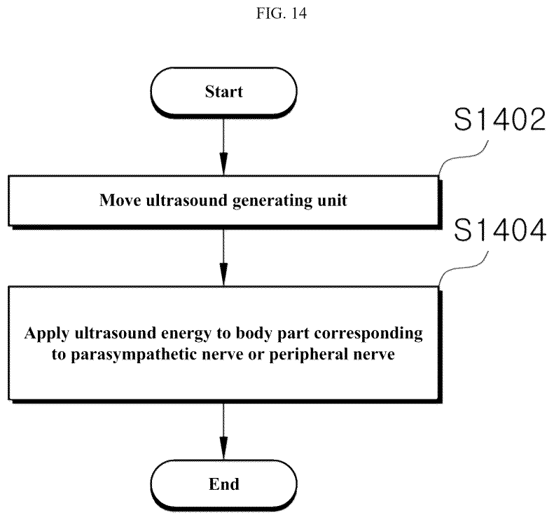

FIG. 14 is a flowchart showing an example of an ultrasound generating method of an ultrasound generating device, according to an embodiment of the present disclosure.

Referring to FIG. 14, an ultrasound generating method may include movement step S1402 and control step S1404.

The moving step may move the ultrasound generating unit 110 to irradiate ultrasound onto the parasympathetic nerve (S1402).

The control step may control the ultrasound generating unit 110 such that the ultrasound generating unit 110 applies ultrasound energy to the body part ‘S’ corresponding to at least one of a parasympathetic nerve and a peripheral nerve through the processor 122 when the movement of the ultrasound generating unit 110 is completed (S1404).

Here, the processor 122 may allow the transducer 111 of the ultrasound generating unit 110 to adjust a microvascular blood flow by noninvasively applying ultrasound energy to the body part ‘S’ corresponding to at least one of a parasympathetic nerve and a peripheral nerve. At this time, the processor 122 may allow the transducer 111 of the ultrasound generating unit 110 to adjust a microvascular blood flow by non-invasively applying ultrasound energy to a certain area A1 to A6 . . . , A7 in the depth direction of the body part ‘S’ corresponding to at least one of the parasympathetic nerve and the peripheral nerve. Here, the body part ‘S’ may include at least one of an ear, a neck, an arm, a wrist, a forearm, a hand, a foot, and a leg, and the skin layer of the body part ‘S’ may be epidermis, dermis, subcutaneous fat, and the like.

In this case, the ultrasound generating unit 110 may include an ultrasound sensor that measures the depth of the body part ‘S’, and the processor 122 may determine whether a location of the ultrasound generating unit 110 corresponds to at least one of a preset parasympathetic nerve and a preset peripheral nerve, based on depth information of the body part ‘S’ acquired by an ultrasound sensor. Here, when the location of the ultrasound generating unit 110 corresponds to at least one of the preset parasympathetic nerves and the preset peripheral nerves, the processor 122 may stop the movement of the ultrasound generating unit 110 or may output a notification signal for stopping the movement.

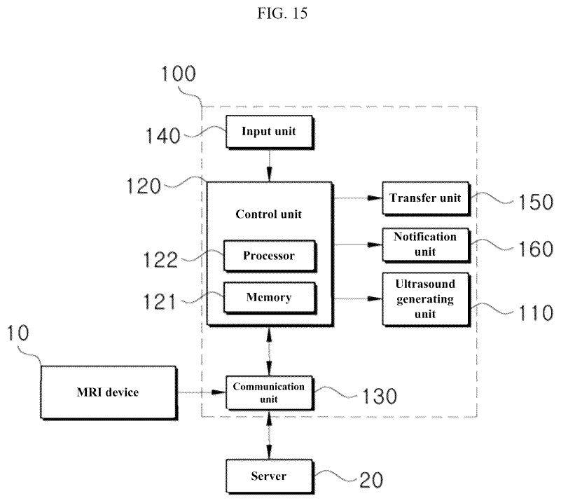

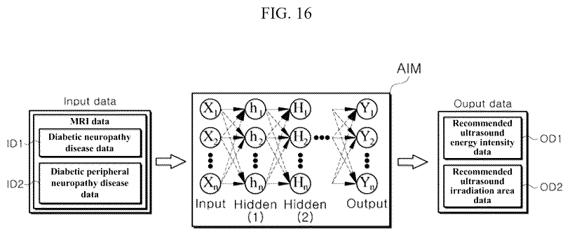

FIG. 15 is a drawing showing another example of a configuration of an ultrasound generating device, according to an embodiment of the present disclosure. FIG. 16 is a diagram illustrating an example of a process for outputting recommended ultrasound energy intensity and a recommended ultrasound irradiation area, which correspond to at least one of a diabetic neuropathy patient and a diabetic peripheral neuropathy patient, through the processor of FIG. 15.

The processor 122 may receive a magnetic resonance image acquired from a magnetic resonance imaging (MRI) device 10 through a communication unit 130, may label diabetic neuropathy disease and diabetic peripheral neuropathy disease in the magnetic resonance image, may perform machine learning-based learning based on at least one of the diabetic neuropathy disease and the diabetic peripheral neuropathy disease, and may output the recommended ultrasound energy intensity and the recommended ultrasound irradiation area by learning based on machine learning. Here, the MRI device 10 may acquire a magnetic resonance image and transmit the acquired magnetic resonance image to the communication unit 130.

In this case, the communication unit 130 may receive the magnetic resonance image acquired from the MRI device 10. Here, the communication unit 130 may include at least one of a wired communication module and a wireless communication module.

Here, in addition to various wired communication modules such as a Local Area Network (LAN) module, a Wide Area Network (WAN) module, or a Value Added Network (VAN) module, the wired communication module may include a variety of cable communication modules such as Universal Serial Bus (USB), High Definition Multimedia Interface (HDMI), Digital Visual Interface (DVI), recommended standard232 (RS-232), power line communication, or plain old telephone service (POTS).

Here, the wireless communication module may include a wireless communication module for supporting various wireless communication methods such as Global System for Mobile (GSM) communication, Code Division Multiple Access (CDMA), Wideband Code Division Multiple Access (WCDMA), Universal Mobile Telecommunication System (UMTS), Time Division Multiple Access (TDMA), Long Term Evolution (LTE), 4G, 5G, and 6G in addition to a Wi-Fi module and Wireless broadband module.

The memory 121 may store the recommended ultrasound energy intensity data and the recommended ultrasound irradiation area data as being learned based on machine learning. Referring to FIG. 16, the processor 122 may learn input values of diabetic neuropathy disease data ID1 and diabetic peripheral neuropathy disease data ID2, which are meta data, based on a machine learning model AIM and then may output result values of recommended ultrasound energy intensity data OD1 and recommended ultrasound irradiation area data OD2. The machine learning model AIM may be built to learn the various pieces of diabetic neuropathy disease data ID1 and the various pieces of diabetic peripheral neuropathy disease data ID2 included in the input data through correlation. The machine learning model AIM may build and reinforce learning through a learning algorithm by using the various pieces of diabetic neuropathy disease data ID1 and the various pieces of diabetic peripheral neuropathy disease data ID2 as a learning data set. At this time, the memory 121 may store the recommended ultrasound energy intensity data OD1 and the recommended ultrasound irradiation area data OD2 as being learned based on the machine learning model AIM.

An input unit 140 may input diabetic neuropathy disease data for a patient with diabetic neuropathy disease, and may input diabetic peripheral neuropathy disease data for a patient with diabetic peripheral neuropathy disease. For example, the input unit 140 may scan and input diabetic neuropathy disease and diabetic peripheral neuropathy disease in the magnetic resonance image, and may be utilized as long as being an input means capable of inputting diabetic neuropathy disease and diabetic peripheral neuropathy disease.

The processor 122 may output the result values of the recommended ultrasound energy intensity data OD1 and the recommended ultrasound irradiation area data OD2, which correspond to the input information of the input unit 140. A transfer unit 150 may be arranged to support the ultrasound generating unit 110 and may move the ultrasound generating unit 110 to the left or right depending on a preset pattern under the control of the processor 122. The ultrasound generating unit 110 having the transducer 111 may irradiate ultrasound onto the body part ‘S’ corresponding to at least one of a parasympathetic nerve and a peripheral nerve in a horizontal direction by movement of the transfer unit 150.

In this case, when irradiating ultrasound onto each irradiation location for each body part ‘S’ corresponding to at least one of the parasympathetic nerve and the peripheral nerve, the ultrasound generating unit 110 may irradiate ultrasound with automatically adjusted energy intensity corresponding to the ultrasound energy intensity data OD1 and an automatically adjusted irradiation area corresponding to the ultrasound irradiation area data OD2, based on the recommended ultrasound energy intensity data OD1 and the recommended ultrasound irradiation area data OD2, which are learned through the correlation between the diabetic neuropathy disease data ID1 and the diabetic peripheral neuropathy disease data ID2.

Accordingly, because automatically adjusting and irradiating ultrasound onto each body part ‘S’ corresponding to at least one of the parasympathetic nerve and the peripheral nerve by using pre-measured information during surgery, the ultrasound generating device 100 according to an embodiment of the present disclosure may irradiate ultrasound onto the irradiation location of the ultrasound, thereby improving the accuracy of the surgery.

Moreover, according to an embodiment of the present disclosure, the ultrasound generating device 100 may control ultrasound for each body part ‘S’ corresponding to at least one of the parasympathetic nerve and the peripheral nerve under optimal conditions in a single surgical procedure, thereby maximizing the surgical effect while reducing the surgical time.

When ultrasound is completely irradiated onto the body part ‘S’ corresponding to at least one of the parasympathetic nerve and the peripheral nerve with the automatically adjusted energy intensity corresponding to the ultrasound energy intensity data OD1 and the automatically adjusted irradiation area corresponding to the ultrasound irradiation area data OD2, the processor 122 may output an irradiation completion status through a notification unit 160. For example, the notification unit 160 may be provided as at least one of a display module and a light emitting diode for visually providing a notification, and may be provided as a speaker or the like for auditorily providing a notification.

The communication unit 130 of the ultrasound generating device 100 according to an embodiment of the present disclosure may further receive a pre-operative image and a post-operative image of each body part. The pre-operative image and the post-operative image of each body part may be acquired through an AI camera. In this case, the communication unit 130 may receive and acquire the pre-operative image and the post-operative image of each body part from a separate device or another server. Here, the processor 122 may further transmit the pre-operative image data and the postoperative image data to a server 20 via the communication unit 130. The server 20 may store the pre-operative image data and the post-operative image data in a database. Users (doctors) of other terminals who share surgical condition information through the server 20 may also utilize the pre-operative image data and the post-operative image data, which are databased, during surgery.

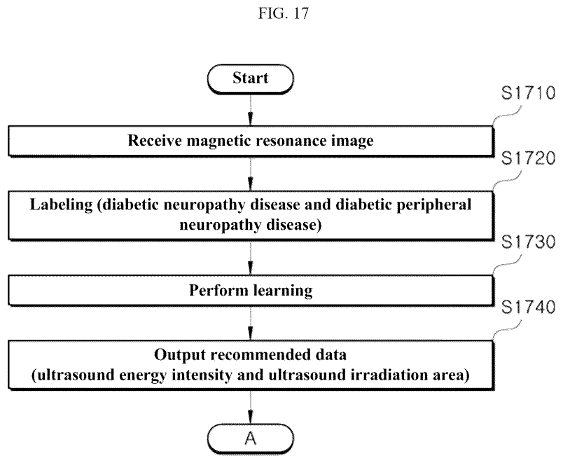

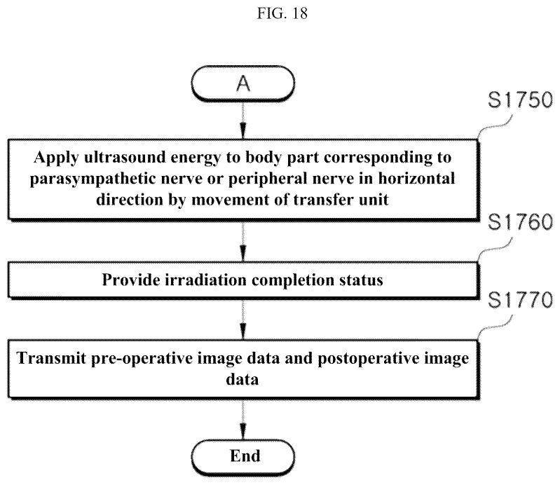

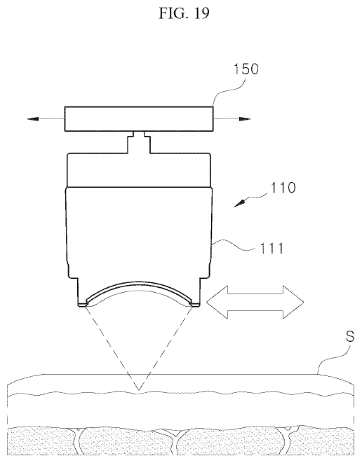

FIGS. 17 and 18 are flowcharts showing another example of an ultrasound generating method of an ultrasound generating device, according to an embodiment of the present disclosure. FIG. 19 is a drawing showing an example of a process of irradiating ultrasound onto a diabetic neuropathy patient or a diabetic peripheral neuropathy patient while an ultrasound generating unit moves by the transfer unit of FIG. 15.

Referring to FIGS. 17 and 18, an ultrasound generating method may include receiving step S1710, labeling step S1720, learning execution step S1730, output step S1740, control step S1750, notification step S1760, and transfer step S1770.

The receiving step may receive the magnetic resonance image acquired from the MRI device 10 through the communication unit 130. In this case, the processor 122 may receive a magnetic resonance image through the communication unit 130 (S1710). Here, the MRI device 10 may acquire a magnetic resonance image and transmit the acquired magnetic resonance image to the communication unit 130.

The labeling step may label diabetic neuropathy disease and diabetic peripheral neuropathy disease in the magnetic resonance image through the processor 122 (S1720).

The learning execution step may perform machine learning-based learning based on at least one of the diabetic neuropathy disease and the diabetic peripheral neuropathy disease through the processor 122 (S1730). The output step may output recommended ultrasound energy intensity and recommended ultrasound irradiation area by learning based on machine learning through the processor 122 (S1740).

The processor 122 may learn input values of the diabetic neuropathy disease data ID1 and the diabetic peripheral neuropathy disease data ID2, which are meta data, based on a machine learning model AIM and then may output result values of the recommended ultrasound energy intensity data OD1 and the recommended ultrasound irradiation area data OD2. The machine learning model AIM may be built to learn the various pieces of diabetic neuropathy disease data ID1 and the various pieces of diabetic peripheral neuropathy disease data ID2 included in the input data through correlation. The machine learning model AIM may build and reinforce learning through a learning algorithm by using the various pieces of diabetic neuropathy disease data ID1 and the various pieces of diabetic peripheral neuropathy disease data ID2 as a learning data set. At this time, the memory 121 may store the recommended ultrasound energy intensity data OD1 and the recommended ultrasound irradiation area data OD2 as being learned based on the machine learning model AIM.

The input unit 140 may input diabetic neuropathy disease data for a patient with diabetic neuropathy disease, and may input diabetic peripheral neuropathy disease data for a patient with diabetic peripheral neuropathy disease. For example, the input unit 140 may scan and input diabetic neuropathy disease and diabetic peripheral neuropathy disease in the magnetic resonance image, and may be utilized as long as being an input means capable of inputting diabetic neuropathy disease and diabetic peripheral neuropathy disease.

The processor 122 may output the result values of the recommended ultrasound energy intensity data OD1 and the recommended ultrasound irradiation area data OD2, which correspond to the input information of the input unit 140 (S1740).

The control step may move the ultrasound generating unit 110 to the left or right depending on a predetermined pattern through the transfer unit 150 under the control of the processor 122. In this case, the ultrasound generating unit 110 having the transducer 111 may move in a horizontal direction by the movement of the transfer unit 150. In this case, the processor 122 may allow the ultrasound generating unit 110 to apply ultrasound energy to the body part ‘S’ corresponding to at least one of a parasympathetic nerve and a peripheral nerve (S1750).

Here, when irradiating ultrasound onto each irradiation location for each body part ‘S’ corresponding to at least one of the parasympathetic nerve and the peripheral nerve, the ultrasound generating unit 110 may irradiate ultrasound with automatically adjusted energy intensity corresponding to the ultrasound energy intensity data OD1 and an automatically adjusted irradiation area corresponding to the ultrasound irradiation area data OD2, based on the recommended ultrasound energy intensity data OD1 and the recommended ultrasound irradiation area data OD2, which are learned through the correlation between the diabetic neuropathy disease data ID1 and the diabetic peripheral neuropathy disease data ID2.

The notification step may output an irradiation completion status through the notification unit 160 by using the processor 122 when ultrasound is completely irradiated the body part ‘S’ corresponding to at least one of the parasympathetic nerve and the peripheral nerve with the automatically adjusted energy intensity corresponding to the ultrasound energy intensity data OD1 and the automatically adjusted irradiation area corresponding to the ultrasound irradiation area data OD2 (S1760). For example, the notification unit 160 may be provided as at least one of a display module and a light emitting diode for visually providing a notification, and may be provided as a speaker or the like for auditorily providing a notification.

The transfer step may further receive a pre-operative image and a post-operative image of each body part through the communication unit 130. Here, the pre-operative image and the post-operative image of each body part may be acquired through an AI camera. In this case, the communication unit 130 may receive and acquire the pre-operative image and the post-operative image of each body part from a separate device or another server. Here, the processor 122 may further transmit the pre-operative image data and the postoperative image data to the server 20 via the communication unit 130 (S1770). The server 20 may store the pre-operative image data and the post-operative image data in a database. Users (doctors) of other terminals who share surgical condition information through the server 20 may also utilize the pre-operative image data and the post-operative image data, which are databased, during surgery.

In the present disclosure, ‘pain’, which appears as a symptom of diabetic peripheral neuropathy, causes direct suffering to a patient, and ‘microcirculation changes’ mediate the worsening of nerve damage.

Accordingly, the present disclosure may relieve pain by noninvasively delivering ultrasound energy to a parasympathetic nerve by using a transducer to adjust the parasympathetic nerve. In other words, pain and the neuropathic changes that cause the pain are adjusted by an autonomic nervous system, thereby reducing symptoms by activating the parasympathetic nerve. Accordingly, the parasympathetic nerve may be activated by applying ultrasound stimulation to the vagus nerve branches, not extremities where pain is felt, thereby alleviating pain and neurodegeneration. Moreover, neuropathic pain may be alleviated by stimulating the parasympathetic nerve system through transcutaneous auricular vagus nerve stimulation (taVNS). That is, the present disclosure may inhibit the development of diabetic neuropathy and may help relieve pain by improving a serotonin function.

The present disclosure may treat microvascular dysfunction in diabetic patients by noninvasively delivering ultrasound energy to peripheral nerves by using a transducer to adjust a microvascular blood flow. That is, the microvascular dysfunction and hyperglycemia in diabetic states form a vicious cycle and may worsen neuropathy in the extremities. Accordingly, the worsening of the condition may be prevented by adjusting a microvascular blood flow by applying ultrasound stimulation to peripheral nerves.

The present disclosure simultaneously delivers ultrasound energy to a certain area in a depth direction of the target nerve, and thus it may be used universally without the need to adjust the irradiation depth for each patient.

Meanwhile, the present disclosure may alleviate symptoms of other diseases through ultrasound irradiation in addition to diabetes and peripheral nerve disease. For example, the other diseases include: nerve compression, entrapment, or laceration (e.g., crutches, ulnar neuropathy, thoracic outlet syndrome, dysesthesia myalgia, Morton's metatarsalgia); other metabolic disorders (e.g., hypothyroidism); autoimmune disorders (e.g., lupus, rheumatoid arthritis, Guillain-Barré syndrome, Miller Fisher syndrome); kidney disease, liver disease, toxin-induced neuropathy (e.g., alcohol, tobacco, asbestos, arsenic, lead, or mercury-related); malignant lymphoma; lung cancer; viral or bacterial infections (e.g., HIV, Lyme disease, leprosy, poliomyelitis); drug-induced neuropathy (chemotherapy); trauma; carpal tunnel syndrome; cubital tunnel syndrome; and vitamin deficiencies (e.g., vitamin B deficiency). Genetic causes of peripheral nerve pathology include, but are not limited to, Charcot-Marie-Tooth disease, Kennedy disease (X-linked spinal muscular atrophy), Van Allen syndrome (hereditary amyloid neuropathy), Refsum disease, and Tangier disease.

At least one component may be added or deleted to correspond to the performance of the components illustrated in FIGS. 1 and 15. Furthermore, it will be easily understood by those skilled in the art that mutual locations of the components may be changed to correspond to the performance or structure of the system.

FIGS. 14, 17, and 18 illustrate that operations are performed sequentially. However, this is merely illustrative of the technical idea of the inventive concept. Those skilled in the art to which an embodiment of the inventive concept belongs may apply various modifications and variations by changing and performing the order illustrated in FIGS. 14, 17, and 18 or performing one or more operations among a plurality of operations in parallel without departing from the essential characteristics of an embodiment of the inventive concept. The embodiment in FIGS. 14, 17, and 18 is not limited to a time-series order.

Meanwhile, the disclosed embodiments may be implemented in a form of a recording medium storing instructions executable by a computer. The instructions may be stored in a form of program codes, and, when executed by a processor, generate a program module to perform operations of the disclosed embodiments. The recording medium may be implemented as a computer-readable recording medium.

The computer-readable recording medium may include all kinds of recording media in which instructions capable of being decoded by a computer are stored. For example, there may be read only memory (ROM), random access memory (RAM), magnetic tape, magnetic disk, flash memory, optical data storage device, and the like.

Disclosed embodiments are described above with reference to the accompanying drawings. One ordinary skilled in the art to which the present disclosure belongs will understand that the present disclosure may be practiced in forms other than the disclosed embodiments without altering the technical ideas or essential features of the present disclosure. The disclosed embodiments are examples and should not be construed as limited thereto.

According to the above-mentioned problem solving means of the present disclosure, a device may accurately irradiate ultrasound onto each body part to improve the accuracy of surgery, thereby suppressing at least one of diabetic neuropathy and diabetic peripheral neuropathy.

Moreover, according to the above-mentioned problem solving means of the present disclosure, a device may control and irradiate ultrasound for each body part under optimal conditions, thereby maximizing the surgical effect while reducing the surgical time.

Effects of the present disclosure are not limited to the effects mentioned above, and other effects not mentioned will be clearly understood by those skilled in the art from the following description.

While the present disclosure has been described with reference to embodiments, it will be apparent to those skilled in the art that various changes and modifications may be made without departing from the spirit and scope of the present disclosure. Therefore, it should be understood that the above embodiments are not limiting, but illustrative.

What is claimed is:

1. An ultrasound generating device comprising:
an ultrasound generating unit configured to irradiate ultrasound onto a parasympathetic nerve; and
a control unit configured to control an operation of the ultrasound generating unit,
wherein the control unit is configured to:
control the ultrasound generating unit such that the ultrasound generating unit applies ultrasound energy to a body part corresponding to the parasympathetic nerve.

2. The ultrasound generating device of claim 1, wherein the control unit is configured to:
control a transducer of the ultrasound generating unit such that the ultrasound generating unit adjusts a microvascular blood flow by noninvasively applying ultrasound energy to a body part corresponding to a peripheral nerve.

3. The ultrasound generating device of claim 2, wherein the control unit is configured to:
control the transducer of the ultrasound generating unit such that the ultrasound generating unit noninvasively applies ultrasound energy to a certain area in a depth direction of the body part corresponding to the parasympathetic nerve or the peripheral nerve.

4. The ultrasound generating device of claim 3, wherein the transducer is in contact with a body part of a user having at least one of a flat surface and a curved surface.

5. The ultrasound generating device of claim 4, wherein the body part includes at least one of an ear, a neck, an arm, a wrist, a forearm, a hand, a foot, and a leg.

6. A method performed by an ultrasound generating device, the method comprising:
moving an ultrasound generating unit to irradiate ultrasound onto a parasympathetic nerve; and
controlling the ultrasound generating unit such that the ultrasound generating unit applies ultrasound energy to a body part corresponding to the parasympathetic nerve when movement of the ultrasound generating unit is completed.

7. The method of claim 6, wherein the controlling includes:
controlling a transducer of the ultrasound generating unit such that the ultrasound generating unit adjusts a microvascular blood flow by noninvasively applying ultrasound energy to a body part corresponding to a peripheral nerve.

8. The method of claim 7, wherein the controlling includes:
controlling the transducer of the ultrasound generating unit such that the ultrasound generating unit noninvasively applies ultrasound energy to a certain area in a depth direction of the body part corresponding to the parasympathetic nerve or the peripheral nerve.

9. The method of claim 8, wherein the transducer is in contact with a body part of a user having at least one of a flat surface and a curved surface.

10. A program stored in a computer-readable recording medium in combination with a computer, which is hardware, to perform the method performed by the ultrasound generating device of claim 6.
